(19) United States
(12) Patent Application Publication
(10) Pub. No.: US 2013/0261058 A1
(43) Pub. Date: Oct. 3, 2013

(54) ACCELERATION OF WOUND HEALING BY GROWTH HORMONE RELEASING HORMONE AND ITS AGONISTS

(71) Applicants: Andrew V. Schally, Miami, FL (US); Hippokratis Kiaris, Athens (GR); Norman L. Block, Miami, FL (US)

(72) Inventors: Andrew V. Schally, Miami, FL (US); Hippokratis Kiaris, Athens (GR); Norman L. Block, Miami, FL (US)

(73) Assignees: University of Miami, Miami, FL (US); U.S. Department of Veterans Affairs, Washington, DC (US); National and Kapodistrian University of Athens, Athens (GR)

(21) Appl. No.: 13/823,729

(22) PCT Filed: Sep. 16, 2011
(86) PCT No.: PCT/US2011/052031

§ 371 Date: Mar. 14, 2013

Related U.S. Application Data
(60) Provisional application No. 61/383,507, filed on Sep. 16, 2010.

(51) Int. Cl. A61K 38/25; A61K 38/16

(52) U.S. Cl. CPC A61K 38/25; A61K 38/16
USPC 514/11.2

(57) ABSTRACT

Agonists of growth hormone releasing hormone promote islet graft growth and proliferation in patients. Methods of treating patients comprise the use of these agonists.

### STATEMENT REGARDING FEDERALLY SPONSORED RESEARCH

Dr. Andrew Schally is an employee of the United States government. This invention was made in part with Government support from the Medical Research Service of the Veterans Affairs Department. The U.S. government may have certain rights in the invention.

### FIELD OF THE INVENTION

Embodiments of the invention provide for methods of accelerating wound healing by growth hormone releasing hormone (GHRH) and its agonists.

### BACKGROUND

Growth hormone releasing hormone (GHRH) is produced by the hypothalamus and acts on the pituitary stimulating the production and release of growth hormone (GH) (1). Besides its hypophyseal action, GHRH may play a role in extrapituitary tissues and importantly in cancers stimulating tumor growth by paracrine and/or autocrine mechanisms (2, 3). Conversely, antagonistic analogs of GHRH inhibit the growth of various experimental human cancers in vitro and in vivo, suggesting that GHRH antagonists could serve as anticancer agents (4).

In addition to the hypothalamus, expression of GHRH has been reported in several non-hypothalamic tissues including placenta, ovaries, testes, lymphocytes and others (5-14), but the physiological significance of this ectopic production of GHRH remains unclear. Locally produced GHRH may promote follicular maturation by paracrine modulation of the stimulatory action of follicle stimulating hormone on granulosa cell function (12). In Leydig cells, GHRH contributes to spermatogenesis (9).

The extrapituitary effects of GHRH in peripheral tissues, including cancers, are mediated, at least in part, by the splice variant of GHRH receptor (SV1). SV1 is derived by the alternative splicing of the RNA encoding the pituitary GHRH receptor (18). Contrary to the relatively restricted pattern of expression of GHRH receptor, SV1 is expressed in several extrapituitary tissues, including cancers, and exhibits both ligand-dependent and ligand-independent activity (19, 20).

### SUMMARY

Embodiments of the invention include administration of a therapeutically effective amount of growth hormone releasing hormone (GHRH), agonist of GHRH, or combinations thereof to a patient, for example, for treating wounds, tissue and/or structure repair, and/or wound healing. The wounds can be due to surgery, traumatic injury, disease, or another cause.

In an embodiment of the invention, a method of treating wounds comprises exposing the cells, structures, and/or tissues to a therapeutically effective amount of growth hormone releasing hormone (GHRH), or an agonist of growth hormone releasing hormone (GHRH), or combinations thereof. In a preferred embodiment the cells comprise fibroblast cells. In an embodiment, an agonist of GHRH comprises a peptide set forth as SEQ ID NO: 1 and/or SEQ ID NO: 2, below.

A method according to the invention for accelerating healing of, repair of, and/or regeneration in a wound in vivo includes exposing cells, structures, and/or tissues to a therapeutically effective amount of growth hormone releasing hormone (GHRH) and/or at least one agonist of growth hormone releasing hormone (GHRH). The healing of, repair of, and/or regeneration in a wound can be accelerated as compared to a control lacking treatment with GHRH and/or GHRH agonist. For example, dermal reestablishment, epidermis tensile strength, epidermal thickness, re-epithelialization, and/or adherence of a skin graft in the wound can be promoted. The cells, structures, and/or tissues can be exposed to a therapeutically effective amount of tesamorelin.

The agonist of GHRH can include a peptide set forth as SEQ ID NO: 1, including Q¹-CO—R²—R³-Ala⁴-Ile⁵-Phe⁶-Thr⁷-R⁸-Ser⁹-Tyr¹⁰-Arg¹¹-R¹²—R¹³-Leu¹⁴-R¹⁵-Gln¹⁶-Leu¹⁷-Ser¹⁸-Ala¹⁹-Arg²⁰-R²¹—R²²—R²³-Gln²⁴-R²⁵Ile²⁶-R²⁷—R²⁸—NH-Q²,

wherein Q¹ is an omega or alpha-omega substituted alkyl having a structure

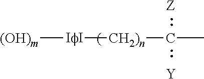

and wherein the following are the case: IφI is phenyl; Y is H, —NH₂, CH₃CONH— or CH₃NH—; Z is H or CH₃; m is 1 or 2 and n is 0, 1 or 2; R² is Ala, Abu or Aib; R³ is Asp or Glu; R⁸ is Asn, Ser, Gln or Thr; R¹² is Lys or Orn; R¹³ is Val or Ile; wherein R¹⁵ is Ala, Gly or Abu; R²¹ is Lys or Orn; R²² is Leu, Ala or Abu; R²³ is Leu, Ala or Abu; R²⁵ is Asp or Glu; R²⁷ is Met, Nle, Ile, or Leu; R²⁸ is Asp, Asn or Ser; Q² is a lower omega-guanidino-alkyl group having a formula (CH₂)ₚ—NH—C(NH₂)═NH; and p is 2-6. The agonist of GHRH can include one or more pharmaceutically acceptable salts of such peptide.

The agonist of GHRH can include a peptide set forth as SEQ ID NO: 2, including Q¹-CO-Ala²-Asp³-Ala⁴-Ile⁵-Phe⁶-Thr⁷-R⁸-Ser⁹-Tyr¹⁰-Arg¹¹-R¹²-Val¹³-Leu¹⁴-R¹⁵-Gln¹⁶-Leu¹⁷-Ser¹⁸-Ala¹⁹-Arg²⁰-R²¹-Leu²²-Leu²³-Gln²⁴-Asp²⁵-Ile²⁶-R²⁷—R²⁸—NH-Q²,

wherein Q¹-CO is Dat,
and wherein the following are the case: R⁸ is Asn, Ser, Gln or Thr; R¹⁵ is Abu; at least one of R¹² and R²¹ is Orn; R²⁷ is Met or Nle; R²⁸ is Ser or Asp; and NH-Q² is Agm. The agonist of GHRH can include one or more pharmaceutically acceptable salts of such peptide.

The agonist of GHRH can include at least one of the following:

JI-34 [Dat¹, Orn^{12,21}, Abu¹⁵, Nle²⁷, Asp²⁸, Agm²⁹]hGH-RH(1-29)NH₂, that is, a peptide having the formula Dat¹-Ala²-Asp³-Ala⁴-Ile⁵-Phe⁶-Thr⁷-Asn⁸-Ser⁹-Tyr¹⁰-Arg¹¹-Orn¹²-Val¹³-Leu¹⁴-Abu¹⁵-Gln¹⁶-Leu¹⁷-Ser¹⁸-Ala¹⁹-Arg²⁰-Orn²¹-Leu²²-Leu²³-Gln²⁴-Asp²⁵-Ile²⁶-Nle²⁷-Asp²⁸-NH—(CH₂)₄—NH—C(NH₂)═NH;

JI-36 [Dat¹, Thr⁸, Orn^{12,21}, Abu¹⁵, Nle²⁷, Asp²⁸, Agm²⁹]hGH-RH(1-29)NH₂, that is, a peptide having the formula Dat¹-Ala²-Asp³-Ala⁴-Ile⁵-Phe⁶-Thr⁷-Thr⁸-Ser⁹-Tyr¹⁰-Arg¹¹-Orn¹²-Val¹³-Leu¹⁴-Abu¹⁵-Gln¹⁶-Leu¹⁷-Ser¹⁸-Ala¹⁹-Arg²⁰-Orn²¹-Leu²²-Leu²³-Gln²⁴-Asp²⁵Ile²⁶-Nle²⁷-Asp²⁸—NH(CH₂)₄—NH—C(NH₂)═NH; and

JI-38 [Dat¹, Gln⁸, Orn^{12,21}, Abu¹⁵, Nle²⁷, Asp²⁸, Agm²⁹]hGH-RH(1-29)NH₂, that is, a peptide having the formula Dat¹-Ala²-Asp³-Ala⁴-Ile⁵-Phe⁶-Thr⁷-Gln⁸-Ser⁹-Tyr¹⁰-Arg¹-Orn¹²-Val¹³-Leu¹⁴-Abu¹⁵-Gln¹⁶-Leu¹⁷-Ser¹⁸-Ala¹⁹-Arg²⁰-Orn²¹-Leu²²-Leu²³-Gln²⁴-Asp²⁵-Ile²⁶-Nle²⁷-Asp²⁸—NH—(CH₂)₄—NH—C(NH₂)═NH.

The wound to be treated can include one or more of the following: a surgical wound, an excisional wound, a deep wound, a skin graft, organ transplantation, tissue or organ damage, a soft tissue injury, a muscle tear, an eye tissue wound, a dental tissue wound, an oral cavity wound, a wound and/or ulcer of the gastro-intestinal mucosa, a diabetic ulcer, a dermal ulcer, a cubitus ulcer, an arterial ulcer, a venous stasis ulcer, a venous leg ulcer, a wound associated with ischemia and ischemic injury, a mechanical wound, an incision wound, a puncture wound, a tear wound, an abrasion wound, a projective wound, a burn, a thermal wound, a chemical wound, a radiation wound, a congestion-related wound, a traumatic wound, and/or a diabetes induced wound.

For example, the wound and/or healing of, repair of; and/or regeneration in the wound can be effected or induced by uremia, malnutrition, vitamin deficiency, obesity, infection, immunosuppression, disease, a steroid, radiation therapy, an antineoplastic drug, an antimetabolite, a pharmaceutical, ischemia and/or ischemic injury.

A method according to the invention for promoting cellular, structural, and/or tissue regeneration in vivo includes exposing cells, structures, and/or tissues to a therapeutically effective amount of growth hormone releasing hormone (GHRH) and/or at least one agonist of growth hormone releasing hormone (GHRH). The cellular, structural, and/or tissue regeneration can be promoted as compared to a control lacking treatment with GHRH and/or GHRH agonist.

A method according to the invention for inducing cell proliferation, cell migration, and/or smooth muscle actin-alpha (αSMA) expression in vitro and/or in vivo includes exposing cells, structures, and/or tissues in vitro and/or in vivo to an effective amount of growth hormone releasing hormone (GHRH) and/or at least one agonist of growth hormone releasing hormone (GHRH).

In an embodiment according to the invention, growth hormone releasing hormone (GHRH) and/or an agonist of growth hormone releasing hormone (GHRH) is used in the manufacture of a medicament for use in a method for accelerating wound healing and/or repair in vivo. In an embodiment according to the invention, growth hormone releasing hormone (GHRH) and/or an agonist of growth hormone releasing hormone (GHRH) is used in the manufacture of a medicament for use in a method for treating wounds. In an embodiment according to the invention, growth hormone releasing hormone (GHRH) and/or an agonist of growth hormone releasing hormone (GHRH) is used in the manufacture of a medicament for use in a method for promoting cellular, structural, and/or tissue regeneration in vivo.

In an embodiment according to the invention, a pharmaceutical composition includes a therapeutically effective amount of growth hormone releasing hormone (GHRH) and/or an agonist of growth hormone releasing hormone (GHRH) for use in a method for accelerating wound healing and/or repair in vivo. In an embodiment according to the invention, a pharmaceutical composition includes a therapeutically effective amount of growth hormone releasing hormone (GHRH) and/or an agonist of growth hormone releasing hormone (GHRH) for use in a method for treating wounds. In an embodiment according to the invention, a pharmaceutical composition includes a therapeutically effective amount of growth hormone releasing hormone (GHRH) and/or an agonist of growth hormone releasing hormone (GHRH) for use in a method for promoting cellular, structural, and/or tissue regeneration in vivo.

Other aspects are described infra.

### BRIEF DESCRIPTION OF THE DRAWINGS

FIG. 1. Expression of SV1 in fibroblasts. A: Western blot analysis of MEFs cultured for 24 h in the presence of GHRH or 51-38 and blotted for SV1, SMA and FAK. Results showed that 100 nM GHRH and the GHRH agonist JI-38 induced SMA and FAK expression in MEFs. β-actin was used as a loading control. B: Immunohistochemical analysis of expression of the GHRH receptor SV1 in mouse 4 mm skin wounds, 5 days after skin incision. Results showed a mosaic pattern of anti-SV1 immunoreactivity as evidenced by the brown precipitant (arrows).

FIG. 2. βGal staining in lung fibroblasts from transgenic mice bearing an αSMA promoter-βGal reporter transgene, exposed to GHRH or 51-38 at 100 nM and 500 nM. A: βGal staining (blue) shows a mosaic pattern of positivity implying the induction of αSMA in a fraction of the fibroblasts. B: Graphical presentation of the βGal-positive fibroblasts indicates that GHRH and 51-38 treatment resulted in a nearly 3-fold increase in the fraction of αSMA-lacZ positive cells.

FIG. 3. GHRH and 51-38 induce migration of cultured MEFs. A. Scratch migration assay of MEFs exposed to 100 nM GHRH or 51-38 showed increased migration of cells as compared to controls. B. GHRH (100 nM) and 51-38 (100 nM and 500 nM) significantly increased migration of MEFs in an 8 μm-pore transwell system. C. GHRH stimulated proliferation of MEFs but only at a concentration of 100 nM. *, p<0.01; **, p<0.001.

FIG. 4. Healing of 4 mm circular skin wounds in mice exposed to GHRH or GHRH agonist 51-38 twice per day at 100 nM. Four wounds were generated in each mouse and always included controls, exposed to vehicle alone. Time points and treatment regimens are indicated. FIG. 4A: Representative microphotographs of control, GHRH and 51-38 treated wounds on days 0, 5 and 10 post incisions. Relative wound area is shown in the graph (FIG. 4B). Experimental groups for wound area measurements consisted of 5, 7 and 5 wounds respectively for the control, GHRH and JI-38 treated wounds. Data are expressed as mean±SEM of % wound area relative to wound area on day 0. *, p<0.05.

FIG. 5. Histology of 4 mm circular skin wounds in mice exposed to GHRH or GHRH agonist 51-38 twice per day at 100 nM. At day 5, a considerable increase in the fibroblasts was observed in the GHRH and JI-38-treated mice, as opposed to edematous and loose stroma of the controls. At day 8, the epidermis has been regenerated in all 3 cases. In GHRH-treated wounds the epidermis, as well as the dermis were almost normal with a diffuse mild inflammatory infiltration. In JI-38 treated wounds the stroma is dense and rich in fibroblasts indicative for a more advanced stage of healing as compared to the control. Van Gieson staining at day 8 (FIG. 5) indicated the nearly normally arranged collagen fibers in the lamina propria of the GHRH-treated samples. In the controls and JI-38 treated specimens, collagen fibers (red) could not be seen, implying delayed healing. Collagen fibers in the bottom of these sections can be seen and correspond to the un-wounded (normal) epithelium s, stroma; e, epithelium; c, collagen fibers stained red by the Van Gieson staining.

FIG. 6. Quantification of fibroblast content in 4 mm circular skin wounds in mice exposed to GHRH or GHRH agonist JI-38 twice per day at 100 nM on day 5 and 8. Fibroblast number was assessed by direct counting of fibroblast in at least 3 random optic fields in the wound areas under light microscope and expressed relative to the fibroblast content in the controls on day 5. Data are expressed as mean±SEM. *, p<0.05.

### DETAILED DESCRIPTION

The present invention is described with reference to the attached Figures, wherein like reference numerals are used throughout the Figures to designate similar or equivalent elements. The Figures are not drawn to scale, and they are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. All references cited herein are incorporated by reference as if each had been individually incorporated.

This application claims the benefit of U.S. Provisional Application No. 61/383,507, which is hereby incorporated by reference in its entirety. All documents cited herein are hereby incorporated by reference as if each had been individually incorporated.

In this document, an amino acid may be indicated by its standard three-letter abbreviation and/or its standard one-letter abbreviation, as understood by one of ordinary skill in the art. For example, a polypeptide sequence may be represented by a string of three-letter abbreviations or by a string of letters.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### DEFINITIONS

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, a “target cell” or “recipient cell” refers to an individual cell or cell which is desired to be, or has been, a recipient of agonists of growth hormone releasing hormone (GHRH). The term is also intended to include progeny of a single cell.

As used herein, “analog” of polypeptides refers to an amino acid sequence that is altered by one or more amino acid residues. The analog may have “conservative” changes, wherein a substituted amino acid has similar structural or chemical properties (e.g., replacement of leucine with isoleucine). More rarely, an analog may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

By the term “modulate,” it is meant that any of the mentioned activities, are, e.g., increased, enhanced, increased, agonized (acts as an agonist), promoted, decreased, reduced, suppressed blocked, or antagonized (acts as an antagonist). Modulation can increase activity more than 1-fold, 2-fold, 3-fold, 5-fold, 10-fold, 100-fold, etc., over baseline values. Modulation can also decrease its activity below baseline values. Modulation can also normalize an activity to a baseline value.

As used herein, a “pharmaceutically acceptable” component/carrier etc is one that is suitable for use with humans and/or animals without undue adverse side effects (such as toxicity, irritation, and allergic response) commensurate with a reasonable benefit/risk ratio.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, a “pharmaceutical salt” includes, but is not limited to, a mineral or organic acid salt of basic residues such as amines, and an alkali or organic salt of acidic residues such as carboxylic acids. Preferably the salt is made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like. The most preferred salt is the hydrochloride salt.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient”, “individual”, and “subject” are used interchangeably herein, and refers to a mammal to be treated, with humans being preferred. In some cases, the methods of the invention find use in experimental animals, in veterinary application, and in the development of animal models for disease, including, but not limited to, rodents including mice, rats, and hamsters; and primates.

“Treatment” or “therapeutic treatment” is an intervention performed with the intention of altering the pathology or symptoms of a disorder. “Treatment” may also be specified as palliative care. Accordingly, “treating” or “treatment” of a state, disorder or condition includes: (1) preventing or delaying the appearance of clinical symptoms of the state, disorder or condition developing in a human or other mammal that may be afflicted with the state, disorder or condition but does not yet experience or display clinical or subclinical symptoms of the state, disorder or condition; (2) inhibiting the state, disorder or condition, i.e., arresting, reducing or delaying the development of the disease or a relapse thereof (in case of maintenance treatment) or at least one clinical or subclinical symptom thereof; or (3) relieving the disease, i.e., causing regression of the state, disorder or condition or at least one of its clinical or subclinical symptoms. The benefit to an individual to be treated is either statistically significant or at least perceptible to the patient or to the physician.

“Prevention” is an intervention performed with the intention of preventing the development of a disorder. “Prevention” refers to prophylactic or preventative measures. Those in need of prevention include those in which the disorder is to be prevented, for example, a human or other mammal that may be predisposed to the disorder. The benefit to an individual in which the disorder is to be prevented is either statistically significant or at least perceptible to the patient or to the physician.

As used herein, “wound healing” refers to the physiological processes for regenerating damaged tissue and for closing a wound, especially formation of new connective tissue and capillaries. The wound healing may be primary wound healing (first intention healing), which is characterized by rapid and complication-free closure and substantially complete recovery as a result of minimal formation of new connective tissue between the edges of a wound, which have a good blood supply and are approximated where appropriate, of a clean wound. Wounds where the edges of the wound are further apart and, in particular, crushed or necrotic, and infected wounds, undergo delayed secondary wound healing in which, as a result of for example, bacterial inflammation, there is filling of the tissue defect with granulation tissue and extensive formation of scar tissue. Epithelialization starting from the edge terminates the wound healing.

As used herein, “accelerated wound healing” refers to the healing when the cells, structures, and/or tissues are exposed to GHRH and/or GHRH agonists as compared to a control in the absence of GHRH and/or GHRH agonists.

As used herein, the term “wound” refers to any damage sustained by cells or tissues and is an interruption of the coherence of body tissues with or without loss of substance and caused by mechanical injury or physically caused cell damage. Types of wounds include mechanical wounds, thermal wounds, chemical wounds, radiation wounds and disease-related wounds. Mechanical wounds can arise through traumatic violence and can occur, for example, as incision and puncture wounds, crushing, lacerating, tearing and abrading wounds, scratch and bite wounds and projective wounds. Thermal wounds arise through exposure to heat or cold. Chemical wounds can arise through the action of acids or alkalis. Radiation wounds arise, for example, through exposure to actinic and/or ionizing radiation, such as ultraviolet light, X-rays, alpha rays, beta rays, and gamma rays. Wounds occurring in relation to disease include, for example, congestion-related wounds, traumatic wounds, diabetes induced wounds, etc.

### GHRH and Agonists of GHRH

Recent evidence demonstrates that GHRH agonists can induce cardiac repair following myocardial infarction, by mechanisms involving a direct action on the cardiomyocytes (15, 16). GHRH agonists can stimulate the proliferation and reduce apoptosis of pancreatic islet cells (17).

Normal human fibroblasts respond to agonistic analogs of GHRH by increased proliferation, an effect that was apparent only in early passage cultures of fibroblasts and was abolished as the cultured cells approached senescence (21). These findings are consistent with a report that GHRH antagonists inhibit telomerase activity (22). Given the responsiveness of fibroblasts to GHRH agonists and recent evidence suggesting a role for GHRH agonists in cell migration, the hypothesis that GHRH agonists play a role in promoting wound healing, tissue repair and tissue regeneration was tested. Wound healing, tissue repair and tissue regeneration is a complex process in which concerted proliferation, migration and re-organization of fibroblasts play an essential role (25). The results herein, provide evidence for a role of GHRH agonists in wound healing, tissue repair and tissue repair evidencing the clinical application of GHRH agonists in conditions involving skin tissue regeneration.

Thus, in a preferred embodiment, a method of promoting wound healing, tissue repair and tissue regeneration comprises exposing cells, structures, and/or tissues to a therapeutically effective dose of growth hormone releasing hormone or its agonists. In another preferred embodiment, a method of promoting wound healing, tissue repair and tissue regeneration comprises exposing cells, structures, and/or tissues to a therapeutically effective dose of at least one growth hormone releasing hormone agonist, for example JI-38. In another preferred embodiment, a method of promoting wound healing, tissue repair and tissue regeneration comprises exposing cells, structures, and/or tissues to a therapeutically effective dose of growth hormone releasing hormone and/or at least one growth hormone releasing hormone agonist. Various treatment regimens can be followed. For example, a single agonist of GHRH can be administered to a wound (for example, without GHRH itself or a GHRH fragment, e.g., the GHRH(1-29)NH fragment, being administered). For example, GHRH agonist may be administered without GHRH itself, because the GHRH may “saturate” the GHRH receptors which could otherwise be open for the agonist of GHRH. As another example, two or more agonists of GHRH can be administered to a wound (for example, without GHRH itself or a GHRH fragment, e.g., the GHRH(1-29)NH fragment, being administered). As another example, the wounds can be pre-treated with at least one agonist of GHRH, e.g., JI-38, and then GHRH and/or a GHRH analog can be administered. In other preferred embodiments, a therapeutically effective dose of GHRH and/or a GHRH analog is administered to a patient prior to administration of an agonist of GHRH. In another preferred embodiment, a therapeutically effective dose of an agonist of GHRH is administered to a patient concurrently with GHRH and/or a GHRH analog. In other preferred embodiments, the therapeutically effective amounts of GHRH, GHRH analog, agonists of GHRH, or combinations thereof can be administered, for example, prior to tissue injury as in the case of surgical intervention and immediately after the surgery.

Agonist of GHRH and GHRH agonist means a compound other than GHRH which has the function of binding to and stimulating GHRH receptors, resulting in the release of growth hormone, or another physiological, endocrine or cellular response specific for GHRH. A GHRH agonist may comprise a full length GHRH sequence in which certain modifications have been made, e.g., amino acid residues have been substituted, side groups have been added, such as a hexenoyl moiety, or a salt has been formed, such as acetate salt, for example tesamorelin (see, e.g., http://www.rxlist.com/egrifta-drug.htm). Tesamorelin is the acetate salt of a sequence having a hexenoyl moiety attached to the N-terminal part of the GHRH amino acid sequence. The amino acid sequence of GHRH, starting at the N-terminal part is: Tyr¹-Ala²-Asp³-Ala⁴-Ile⁵-Phe⁶-Thr⁷-Asn⁸-Ser⁹-Arg¹¹-Lys¹²-Val¹³-Leu¹⁴-Gly¹⁵-Gln¹⁶-Leu¹⁷-Ser¹⁸-Ala¹⁹-Arg²⁰-Lys²¹-Leu²²-Leu²³-Gln²⁴-Asp²⁵-Ile²⁶-Met²⁷-Ser²⁸-Arg²⁹-Gln³⁰-Gln³¹-Gly³²-Glu³³-Ser³⁴-Asn³⁵-Gln³⁶-Glu³⁷-Arg³⁸-Gly³⁹-Ala⁴⁰-Arg⁴¹-Ala⁴²-Arg⁴³-Leu⁴⁴. A GHRH agonist may comprise a GHRH sequence to which amino acid deletions, insertions, and/or substitutions have been made. A GHRH agonist may also be a fragment or modified fragment of GHRH having the capability to bind to the GHRH receptor and stimulate release of growth hormone. The biological activity of GHRH is understood to reside in the N-terminal 1-29 amino acid sequence of this peptide (12). Thus, fragments or modified fragments between amino acid residues 1 and 29 are expected to be useful.

For example, an agonist of GHRH can include one or more features that protect it against degradation by biological, chemical, and/or other processes. For example, such features can protect the GHRH agonist peptide from proteolytic enzymes in the wound milieu (fluids), e.g., from proteases secreted by neutrophils. Such proteolytic enzymes can inactivate (e.g., degrade or split) unprotected peptides such as unprotected GHRH. Such protective features can include, for example, the replacement of certain amino acids (residues) in the native peptide sequence of GHRH with other different amino acids (residues).

In another preferred embodiment, a method of accelerating wound healing due to traumatic injury, such as injury from burns, in a patient comprises administering to the patient a therapeutically effective amount of GHRH to the burn wounds. In another preferred embodiment, at least one agonist of growth hormone releasing hormone (GHRH) is administered to the wounds of a burn patient. In another preferred embodiment combinations of therapeutically effective amounts of GHRH and agonist of GHRH are administered to the patient. The GHRH, GHRH agonists or combinations thereof, can be administered over periods of time and in varying concentrations.

In another preferred embodiment, the GHRH, GHRH agonist or combinations thereof are used in the pretreatment and/or further treatment of tissues, structures, and/or organs to be transplanted. For example, the transplants are treated with GHRH or GREW agonist or combinations of GHRH and agonist of GHRH before the transplantation, and can be administered immediately before, while still in the donor organism. The recipient organism can likewise be treated with GHRH, GHRH agonist or combinations thereof, from the time of transplantation onwards. This treatment of the organs or tissues to be transplanted, both directly before and after transplantation, is expected to accelerate healing both in the donor and recipient.

In another preferred embodiment, cells, structures, or tissues can be pre-treated in vitro with GHRH, GHRH agonist or combinations thereof, prior to being administered to a patient. The cells can be fibroblasts, tissues which have been cultured ex vivo etc.

In preferred embodiments, the GHRH, GHRH agonist or combinations thereof, accelerate wound healing of wounds including surgical wounds, excisional wounds, deep wounds involving damage of the dermis and epidermis, soft tissue injuries such as muscle tears, eye tissue wounds, dental tissue wounds, oral cavity wounds, wounds and ulcers of the gastro-intestinal mucosa, diabetic ulcers, dermal ulcers, cubitus ulcers, arterial ulcers, venous stasis ulcers, and burns resulting from heat, exposure to extreme temperatures of heat or cold, exposure to radiation, or exposure to chemicals, in normal individuals and those subject to conditions which induce abnormal wound healing such as uremia, malnutrition, vitamin deficiencies, obesity, infection, immunosuppression and complications associated with systemic treatment with steroids, radiation therapy, antineoplastic drugs, antimetabolites, and other drugs and pharmaceuticals. The compositions are also useful for promoting the healing of wounds associated with ischemia and ischemic injury, e.g., chronic venous leg ulcers caused by an impairment of venous circulatory system return and/or insufficiency; for promoting dermal reestablishment subsequent to dermal loss; increasing the tensile strength of epidermis and epidermal thickness; and increasing the adherence of skin grafts to a wound bed and to stimulate re-epithelialization from the wound bed. The compositions can be used to treat acute wounds and/or chronic wounds.

Nomenclature:

The nomenclature used to define the amino acid residues and synthetic peptides is that specified by the IUPAC-IUB Commission on Biochemical Nomenclature (European J. Biochem., 1984, 138, 9-37). By natural amino acid is meant one of the common, naturally occurring amino acids found in proteins comprising Gly, Ala, Val, Leu, Ile, Ser, Thr, Lys, Arg, Asp, Asn, Glu, Gln, Cys, Met, Phe, Tyr, Pro, Trp and His. By Nle is meant norleucine, by Abu is meant alpha amino butyric acid, by Orn is meant ornithine, by Aib is meant alpha iso-butyric acid, by Agm is meant agmatine, by Dat is meant desaminotyrosine, and by Har is meant homoarginine. Other abbreviations used are: Boc- (tert-butyloxycarbonyl-); 2-Br-Cbz (2-bromo-benzyloxycarbonyl-); Cbz- (benzyloxycarbonyl-); Chx- (cyclohexyl-); 2-Cl-Cbz- (2-chloro-benzyloxycarbonyl-); DCCl (dicyclohexylcarbodiimide); DIC (diisopropylcarbodiimide); DCM (dichloromethane); DIEA (diisopropylethylamine); DMF (dimethylformamide); HOBt (1-hydroxybenzotriazole); HPLC (high performance liquid chromatography); MeOH (methyl alcohol); TFA (trifluoroacetic acid); and Tos- (p-toluensulfonyl-). The amino acid sequences of the synthetic peptides are numbered in correspondence with the amino acid residues in hGH-RH(1-29); thus, for example, the Ala⁴ and R⁸ in the synthetic peptides occupy the same position in the sequence as the Ala⁴ and R⁸ residues in hGH-RH(1-29).

The convention under which the N-terminal of a peptide is placed to the left and the C-terminal is placed to the right is followed herein. It should be understood that the terms N- and C-terminal used with respect to the synthetic peptides mean Q¹-CO— and —NH-Q² respectively. Where applicable, standard three-letter abbreviations are used for coded amino acids. Noncoded amino acids and N-acyl moieties are abbreviated as follows: Abu, α-aminobutyric acid; Agm, agmatine; Dat, desaminotyrosine; Har, homoarginine; Nle, norleucine.

Examples of agonists of GHRH have been described; see, for example, U.S. Pat. No. 5,792,747, which is incorporated by reference herein in its entirety. Examples of preferred agonists include synthetic peptides having the sequence set forth as SEQ ID NO: 1 (Formula I):

Q¹-CO—R²—R³-Ala⁴-Ile⁵-Phe⁶-Thr⁷-R⁸-Ser⁹-Tyr¹⁰-Arg¹¹-R¹²—R¹³-Leu¹⁴-R¹⁵-Gln¹⁶-Leu¹⁷-Ser¹⁸Ala¹⁹-Arg²⁰-R²¹—R²²—R²³-Gln²⁴-R²⁵-Ile²⁶-R²⁷—R²⁸—NH-Q²

wherein Q¹ is an omega or alpha-omega substituted alkyl having the structure:

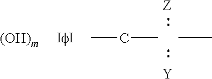

wherein IΦI is phenyl; Y is H, —NH₂, CH₃CONH— or CH₃NH—; Z is H or CH₃; m is 1 or 2 and n is 0, 1 or 2; R² is Ala, Abu or Aib; R³ is Asp or Glu; R⁸ is Asn, Ser, Gln or Thr; R¹² is Lys or Orn; R¹³ is Val or Ile; R¹⁵ is Ala, Gly or Abu; R²′ is Lys or Orn; R²² is Leu, Ala or Abu; R²³ is Leu, Ala or Abu; R²⁵ is Asp or Glu; R²⁷ is Met, Nle, Ile, or Leu; R²⁸ is Asp, Asn or Ser; and, Q² is a lower omega-guanidino-alkyl group having a formula:

(CH₂)ₚ—NH—C(NH₂)═NH

wherein p is 2-6.

In another preferred embodiment, the synthetic peptides comprise the sequence set forth as SEQ ID NO: 2 (Formula II):

Q¹-CO-Ala²-Asp³-Ala⁴-Ile⁵-Phe⁶-Thr⁷-R⁸-Ser⁹-Tyr¹⁰-Arg¹¹-R¹²—Val¹³-Leu¹⁴-R¹⁵-Gln¹⁶-Leu¹⁷-Ser¹⁸-Ala¹⁹-Arg²⁰-R²¹-Leu²²-Leu²³-Gln²⁴-Asp²⁵-Ile²⁶-R²⁷—R²⁸—NH-Q²

wherein Q¹, R⁸, R¹², R¹⁵, R²¹, R²⁷, R²⁸ and Q² are as defined above. Preferably, Q¹-CO is Dat; R⁸ is Asn, Ser, Gln or Thr; R¹⁵ is Abu; at least one of R¹² and R²⁰ is Orn; R²⁷ is Met or Nle; R²⁸ is Ser or Asp; and NH-Q² is Agm. In certain preferred synthetic peptides of Formula II, Q¹-CO is Dat, R¹⁵ is Abu; R²¹ is Orn; R²⁷ is Nle; and NH-Q² forms Agm.

In one preferred agonist, in Q¹, m is 1, n is 1 and Y and Z are H, so that Q¹-CO forms Dat; R¹² is Orn, R¹⁵ is Abu, R²¹ is Orn, R²⁷ is Nle, R²⁸ is Ser; and in Q², p is 4, so that —NH-Q² forms Agm, and the peptide has the formula: Dat¹-Ala²-Asp³-Ala⁴-Ile⁵-Phe⁶-Thr⁷-Asn⁸-Ser⁹-Tyr¹⁰-Arg¹-Orn¹²-Val¹³-Leu¹⁴-Abu¹⁵-Gln¹⁶-Leu¹⁷-Ser¹⁸-Ala¹⁹-Arg²⁰-Orn²¹-Leu²²-Leu²³-Gln²⁴-Asp²⁵-Ile²⁶-Nle²⁷-Ser²⁸-NH—(CH₂)₄—NH—C(NH₂)═NH. This analog may be expressed under a well known convention as follows: (Dat¹, Orn.^{12,21}, Abu¹⁵, Nle²⁷, Agm²⁹)-hGH-RH(1-29).

In another preferred embodiment, an agonist of GHRH comprises a synthetic peptide wherein Q¹-CO— forms Dat, R¹² is Orn, R¹⁵ is Abu, R²¹ is Orn, R²⁷ is Nle, R²⁸ is Asp; and —NH-Q² forms Agm, the peptide has the formula: Dat¹-Ala²-Asp³-Ala⁴-Ile^{s}-Phe⁶-Thr⁷-Asn⁸-Ser⁹-Tyr¹⁰-Arg¹¹-Orn¹²-Val¹³-Leu^{l4}-Abu¹⁵-Gln¹⁶-Leu¹⁷-Ser¹⁸-Ala¹⁹-Arg²⁰-Orn²¹-Leu²²-Leu²³-Gln²⁴-Asp²⁵-Ile²⁶-Nle²⁷-Asp²⁸-NH—(CH₂)₄—NH—C(NH₂)═NH, which may be abbreviated as: (Dat¹, Orn^{12,21}, Abu¹⁵, Nle²⁷, Asp²⁸, Agm²⁹)hGH-RH(1-29); this synthetic peptide is also termed JI-34.

In yet another preferred embodiment, an agonist of GHRH comprises a synthetic peptide wherein Q¹-CO forms Dat, R⁸ is Thr, R¹² is Orn, R¹⁵ is Abu, R²¹ is Orn, R²⁷ is Nle, R²⁸ is Asp; and —NH-Q² forms Agm, the peptide has the formula: Dat¹-Ala²-Asp³-Ala⁴-Ile^{s}-Phe⁶-Thr⁷-Thr⁸-Ser⁹-Tyr¹⁰-Arg¹¹-Orn¹²-Val¹³-Leu¹⁴-Abu¹⁵-Gln¹⁶-Leu¹⁷-Ser¹⁸-Ala¹⁹-Arg²⁰-Orn²¹-Leu²²-Leu²³-Gln²⁴-Asp²⁵-Ile²⁶-Nle²⁷-Asp²⁸-NH(CH₂)₄—NH—C(NH₂)═NH, which may be abbreviated as: (Dat¹, Thr⁸, Orn^{12,21}, Abu¹⁵, Nle²⁷, Asp²⁸, Agm²⁹)hGH-RH(1-29); this synthetic peptide is also termed JI-36.

Similarly, where Q¹-CO is Dat, R⁸ is Gln, R¹² is Orn, R¹⁵ is Abu, R²¹ is Orn, R²⁷ is Nle, R²⁸ is Asp; and Q² forms Agm, the peptide has the formula: Dat¹-Ala²-Asp³-Ala⁴-Ile⁵-Phe⁶-Thr⁷-Gln⁸-Ser⁹-Tyr¹⁰-Arg¹¹-Orn¹²-Val¹³-Leu¹⁴-Abu¹⁵-Gln¹⁶-Leu¹⁷-Ser¹⁸-Ala¹⁹-Arg²⁰-Orn²¹-Leu²²-Leu²³-Gln²⁴-Asp²⁵-Ile²⁶-Nle²⁷-Asp²⁸-NH—(CH₂)₄)—NH—C(NH₂)═NH, which may be abbreviated as: (Dat¹, Gln⁸, Orn^{12,21}, Abu¹⁵, Nle²⁷, Asp²⁸, Agm²⁹)hGH-RH(1-29); this synthetic peptide is also termed JI-38.

### Administration

GHRH and the agonists of GHRH useful in the methods of the invention can be administered by a variety of routes and using pharmaceutical formulations previously developed for other indications. Such delivery routes include, but are not limited to, at least for most known agents, topical delivery, including micelle and nanosphere topical delivery systems, subcutaneous delivery including pump-assisted continuous infusion and disposable micro-pump, inhalation, micro-needles and buccal delivery.

The particular route of administration and pharmaceutical formulation of an agonist used in the practice of the methods of the invention will be selected by the practitioner based on a patient's disease or condition being treated and the agent employed. A wide variety of pharmaceutical compositions can be employed in the methods of the invention. In some embodiments, extended use preparations can be used for ease of administration and increased efficacy.

The present compositions also contemplate wound care dressings or bandages comprising a therapeutically effective amount of GHRH, GHRH agonists, combinations of GHRH and one or more agonists of GHRH in various concentrations, or physiologically acceptable derivatives or salts thereof. The bandage or dressing for wound care can include an outer fabric support, preferably an elastomeric fabric support, and an inner pad, wherein the inner pad includes an outer membrane surface, preferably fabricated from a film-forming material, and incorporating a therapeutically effective amount of the compositions or a physiologically acceptable derivative or salt thereof, in therapeutically effective dosages. The pad may be integral with or separate from the outer fabric support.

The compositions can be incorporated into the membrane matrix, but may also be incorporated into the material of the inner pad contained by the membrane. In addition, the therapeutically active agent, e.g., GHRH, is held in the polymeric matrix, so that migration is inhibited, and the gradual release over time of GHRH compositions is permitted.

In another aspect, the wound dressing comprises an absorbent pad having a liquid pervious body-side liner, a separate outer cover sheet, optionally liquid impervious, and an absorbent body disposed therebetween. The inner and/or absorbent body are fabricated from materials which incorporate a therapeutically effective amount of GHRH, GHRH agonist, or combinations thereof in the matrix or interstitial spaces to ensure that the GHRH compositions are in constant close proximity to the wound. The inner surface or pad of the bandage is preferably fabricated from a natural or synthetic membrane or film-forming material of either organic or inorganic, animal or vegetable origin, or from plastic materials. For example, the inner surface or pad of the bandage can be fabricated from gelatins or from vegetable gums, or from hydrophilic or hydrophobic film forming plastic materials, such as polyvinylchlorides, polyacetates, or polyamides, which are cast or coated as a film or membrane in a conventional or nonconventional way.

Suitable polymeric materials include, but are not limited to, Silastic polymer, silicone-based material, polydimethylsiloxane (PDMS), polyethylene terephthalate (PET), Dacron polymer, knitted Dacron fiber, Dacron velour, polyglactin, nylon, silk, polyethylene (PE), polyurethane, polyvinyl chlorides, Silastic elastomer, silicone, rubber, PMMA (poly-(methylmethacrylate)), latex, polypropylene (PP), polyolefin, cellulose, polyvinyl alcohol (PVA), poly(hydroxyethylmethacrylate) (PHEMA), poly(glycolic acid), poly(acrylonitrile) (PAN), fluorinated ethylene propylene (poly(tetrafluoroethylene-co-hexafluoropropylene)) (FEP), and Teflon polymer (polytetrafluoroethylene) (PTFE), and copolymers thereof, and mixtures, blends, and alloys thereof.

The simplest method of incorporating the therapeutically active compounds into the polymeric material is by direct compounding of the therapeutically active substance into the plastic resin before casting or the like. The film or membrane can be fabricated from a hydrophobic polymer which is both liquid and gas permeable, but impervious to the passage of micro-organisms. The hydrophobicity of the film or membrane can be a useful feature in that it can reduce the tendency for the film or membrane to become attached to the wound site.

The amount of GHRH compositions incorporated into the formulation should only be in a range sufficient to permit ready application of the formulation to the wound area in an amount which will deliver the desired amount of the GHRH compositions, e.g. GHRH, agonists of GHRH, combinations of GHRH and GHRH agonist. The customary amount of formulation to be applied to a wound will depend upon wound size and concentration of GHRH and/or GHRH agonist in the formulation.

The GHRH compositions, e.g., GHRH, GHRH agonist or combinations thereof, may also be administered to the eye to treat lacrimal gland injuries, disorders and pathologies in humans as a liquid, drop, thickened liquid, and/or a gel.

The GHRH compositions can also be intranasally administered to the nasal mucosa to treat disorders, injuries and pathologies of the nasal mucosa and sinus epithelia in humans as liquid drops or in a spray form.

Labels:

The compositions can also be labeled for use both in vitro and in vivo, such as in vitro assays, diagnostics, kits, imaging, therapeutics and the like. In some embodiments, the compounds are detected using a label that provides a labeling signal. In other embodiments, the compounds are labeled with different labeling molecules, so as to differentiate between the compounds. The wording “detectably distinguishable” as used herein with reference to labeling molecules indicates molecules that are distinguishable on the basis of the labeling signal provided by the label compound attached to the molecule. Exemplary label compounds that can be used to provide detectably distinguishable labeling molecules, include but are not limited to radioactive isotopes, fluorophores, chemiluminescent dyes, chromophores, enzymes, enzyme substrates, enzyme cofactors, enzyme inhibitors, dyes, metal ions, nanoparticles, metal sols, ligands (such as biotin, avidin, streptavidin or haptens) and additional compounds identifiable by a skilled person upon reading of the present disclosure.

The terms “label” and “labeling molecule” as used herein as a component of a complex or molecule refer to a molecule capable of detection, including but not limited to radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting fluorescence in a detectable image. As a consequence the wording “labeling signal” as used herein indicates the signal emitted from the label that allows detection of the label, including, but not limited to, radioactivity, fluorescence, chemoluminescence, production of a compound in outcome of an enzymatic reaction and the like.

In imaging uses, the agonists are labeled so that they can be detected outside the body. Typical labels are radioisotopes, usually ones with short half-lives. For example, the usual imaging radioisotopes, such as ¹²³I, ¹²⁴I, ¹²⁵I, ¹³¹I, ^{99m}Tc, ¹⁸⁶Re, ¹⁸⁸Re, ⁶⁴Cu, ⁶⁷Cu, ²¹²Bi, ²¹³Bi, ⁶⁷Ga, ⁹⁰Y, ¹¹¹In, ¹⁸F, ³H, ¹⁴C, ³⁵S or ³²P can be used. Nuclear magnetic resonance (NMR) imaging enhancers, such as gadolinium-153, can also be used to label the agonists for detection by NMR.

Effective Amounts:

The compositions described above are preferably administered to a subject in an effective amount. A therapeutically effective amount is an amount which is capable of producing a desirable result in a treated animal or cell. As is well known in the medical and veterinary arts, dosage for any one animal depends on many factors, including the particular animal's size, body surface area, age, the particular composition to be administered, time and route of administration, general health, and other drugs being administered concurrently. An effective amount for use with a cell in culture will also vary, but can be readily determined empirically (for example, by adding varying concentrations to the cell and selecting the concentration that best produces the desired result). It is expected that an appropriate concentration would be in the range of about 0.001-50 μM. For example, a therapeutically effective amount of GHRH and/or an agonist of growth hormone releasing hormone (GHRH) can be in the range of from about 0.001 μM, 0.01 μM, 0.1 μM, 1 μM, or 10 μM to about 0.01 μM, 0.1 μM, 1 μM, 10 μM, or 50 μM.

Formulations:

A compound of the present invention can be formulated as a pharmaceutical composition. Such a composition can then be administered parenterally, by inhalation spray, rectally, or topically by other means in dosage unit formulations containing conventional nontoxic pharmaceutically acceptable carriers, adjuvants, and vehicles as desired. Topical administration can also involve the use of transdermal administration, such as transdermal patches or iontophoresis devices. The term parenteral as used herein includes subcutaneous injections, intravenous, intramuscular, intrasternal injection, inhalation or infusion techniques.

Suppositories for rectal administration of the drug can be prepared by mixing the drug with a suitable nonirritating excipient such as cocoa butter, synthetic mono- di- or triglycerides, fatty acids and polyethylene glycols that are solid at ordinary temperatures but liquid at the rectal temperature and will therefore melt in the rectum and release the drug.

The methods and combinations of the present invention provide one or more benefits. Combinations of the present invention may allow for a lower dose of each agent. A benefit of lowering the dose of the compounds, compositions, agents and therapies of the present invention administered to a mammal includes a decrease in the incidence of adverse effects associated with higher dosages.

By lowering the incidence of adverse effects, an improvement in the quality of life of a patient undergoing treatment is contemplated. Further benefits of lowering the incidence of adverse effects include an improvement in patient compliance, a reduction in the number of clinical visits needed for the treatment of adverse effects, and a reduction in the administration of analgesic agents needed to treat pain associated with the adverse effects.

Alternatively, the methods and combination of the present invention can also maximize the therapeutic effect at higher doses.

Formulations suitable for topical administration include liquid or semi-liquid preparations suitable for penetration through the skin to the site of where treatment is required, such as liniments, lotions, creams, ointments or pastes, and drops suitable for administration to the eye, ear, or nose. Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified and sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

The composition of the invention can be administered to a patient either alone or in pharmaceutical compositions where it is mixed with suitable carriers or excipient(s). In treating a patient exhibiting a disorder of interest, a therapeutically effective amount of an agent or agents such as these is administered. A therapeutically effective dose refers to that amount of the compound that results in amelioration of symptoms or a prolongation of survival in a patient.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀Compounds which exhibit large therapeutic indices are preferred. The data obtained from these cell culture assays and animal studies can be used in formulating a range of dosage for use in a human. The dosage of such compounds lies preferably within a range of circulating concentrations that include the ED₅₀ with little or no toxicity. The dosage may vary within this range depending upon the dosage form employed and the route of administration utilized.

For any compound used in the method of the invention, the therapeutically effective dose can be estimated initially from cell culture assays. For example, a dose can be formulated in animal models to achieve a circulating plasma concentration range that includes the IC₅₀ as determined in cell culture. Such information can be used to more accurately determine useful doses in humans. Levels in plasma may be measured, for example, by HPLC.

The exact formulation, route of administration and dosage can be chosen by the individual physician in view of the patient's condition (see, e.g., Fingl et al., in The Pharmacological Basis of Therapeutics, 1975, Ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administrated dose in the management of the oncologic or other disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Depending on the specific conditions being treated, such agents may be formulated and administered systemically or locally. Formulations, techniques for formulation, routes of administration, and administration are known in the art, and are described, for example, in Remington's Pharmaceutical Sciences, 18^{th} ed., Mack Publishing Co., Easton, Pa. (1990). Suitable routes may include rectal, transdermal, vaginal, transmucosal, and/or intestinal administration; parenteral delivery, including intramuscular, subcutaneous, intramedullary injections, as well as intrathecal, direct intraventricular, intravenous, intraperitoneal, intranasal, and/or intraocular injections, just to name a few.

The compositions described above may be administered to a subject in any suitable formulation. In addition to treatment with topical formulations of the composition, the composition can be delivered by other methods. For example, the composition can be formulated for parenteral delivery, e.g., for subcutaneous, intravenous, intramuscular, and/or intratumoral injection. Other methods of delivery, for example, liposomal delivery and/or diffusion from a device impregnated with the composition might be used. The compositions may be administered in a single bolus, multiple injections, and/or by continuous infusion (for example, intravenously and/or by peritoneal dialysis). For parenteral administration, the compositions are preferably formulated in a sterilized pyrogen-free form. Compositions of the invention can also be administered in vitro to a cell (for example, to induce apoptosis in a cancer cell in an in vitro culture) by simply adding the composition to the fluid in which the cell is contained.

Depending on the specific conditions being treated, such agents may be formulated and administered systemically or locally. Techniques for formulation and administration may be found in Remington's Pharmaceutical Sciences, 18^{th} ed., Mack Publishing Co., Easton, Pa. (1990). Suitable routes may include oral, rectal, transdermal, vaginal, transmucosal, and/or intestinal administration; parenteral delivery, including intramuscular, subcutaneous, intramedullary injections, as well as intrathecal, direct intraventricular, intravenous, intraperitoneal, intranasal, and/or intraocular injections, just to name a few.

For injection, the agents of the invention may be formulated in aqueous solutions, preferably in physiologically compatible buffers such as Hanks's solution, Ringer's solution, and/or physiological saline buffer. For such transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, and then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to the liposomal hydrophobicity, small organic molecules may be directly administered intracellularly.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, and/or solutions. The pharmaceutical compositions of the present invention may be manufactured in a manner that is itself known, e.g., by means of conventional mixing, dissolving, granulating, dragee-making, levitating, emulsifying, encapsulating, entrapping and/or lyophilizing processes.

Formulations suitable for topical administration include liquid or semi-liquid preparations suitable for penetration through the skin to the site of where treatment is required, such as liniments, lotions, creams, ointments and/or pastes, and/or drops suitable for administration to the eye, ear, and/or nose. Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified and sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogel. The formulation may incorporate any suitable surface active agent such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, and/or sorbitol; cellulose preparations such as, for example, maize starch, wheat starch, rice starch, potato starch, gelatin, gum tragacanth, methyl cellulose, hydroxypropylmethyl-cellulose, sodium carboxy-methylcellulose, and/or polyvinyl pyrrolidone (PVP). If desired, disintegrating agents may be added, such as cross-linked polyvinyl pyrrolidone, agar, and/or alginic acid and/or a salt thereof such as sodium alginate.

Dragee cores are provided with suitable coating. For this purpose, concentrated sugar solutions may be used, which may optionally contain gum arabic, talc, polyvinyl pyrrolidone, carbopol gel, polyethylene glycol, and/or titanium dioxide, lacquer solutions, and/or suitable organic solvents or solvent mixtures. Dyestuffs or pigments may be added to the tablets or dragee coatings for identification or to characterize different combinations of active compound doses.

The composition can include a buffer system, if desired. Buffer systems are chosen to maintain or buffer the pH of compositions within a desired range. The term “buffer system” or “buffer” as used herein refers to a solute agent or agents which, when in a water solution, stabilize such solution against a major change in pH (or hydrogen ion concentration or activity) when acids or bases are added thereto. Solute agent or agents which are thus responsible for a resistance or change in pH from a starting buffered pH value in the range indicated above are well known. While there are countless suitable buffers, potassium phosphate monohydrate is a preferred buffer.

The final pH value of the pharmaceutical composition may vary within the physiological compatible range. For example, the final pH value is one not irritating to human skin and preferably such that transdermal transport of the active compound, for example, growth hormone releasing hormone (GHRH) and/or an agonist of GHRH, is facilitated. Without violating this constraint, the pH may be selected to improve the compound stability and to adjust consistency when required. In one embodiment, the preferred pH value is about 3.0 to about 7.4, more preferably about 3.0 to about 6.5, most preferably from about 3.5 to about 6.0.

For preferred topical delivery vehicles the remaining component of the composition is water, which is necessarily purified, e.g., deionized water. Such delivery vehicle compositions contain water in the range of more than about 50 to about 95 percent, based on the total weight of the composition. The specific amount of water present is not critical, however, being adjustable to obtain the desired viscosity (usually about 50 cps to about 10,000 cps) and/or concentration of the other components. The topical delivery vehicle preferably has a viscosity of at least about 30 centipoises.

Other known transdermal skin penetration enhancers can also be used to facilitate delivery of the composition. Illustrative are sulfoxides such as dimethylsulfoxide (DMSO) and the like; cyclic amides such as 1-dodecylazacycloheptane-2-one (AZONE™, a registered trademark of Nelson Research, Inc.) and the like; amides such as N,N-dimethyl acetamide (DMA) N,N-diethyl toluamide, N,N-dimethyl formamide, N,N-dimethyl octamide, N,N-dimethyl decamide, and the like; pyrrolidone derivatives such as N-methyl-2-pyrrolidone, 2-pyrrolidone, 2-pyrrolidone-5-carboxylic acid, N-(2-hydroxyethyl)-2-pyrrolidone or fatty acid esters thereof, 1-lauryl-4-methoxycarbonyl-2-pyrrolidone, N-tallowalkylpyrrolidones, and the like; polyols such as propylene glycol, ethylene glycol, polyethylene glycol, dipropylene glycol, glycerol, hexanetriol, and the like; linear and branched fatty acids such as oleic, linoleic, lauric, valeric, heptanoic, caproic, myristic, isovaleric, neopentanoic, trimethyl hexanoic, isostearic, and the like; alcohols such as ethanol, propanol, butanol, octanol, oleyl, stearyl, linoleyl, and the like; anionic surfactants such as sodium laurate, sodium lauryl sulfate, and the like; cationic surfactants such as benzalkonium chloride, dodecyltrimethylammonium chloride, cetyltrimethylammonium bromide, and the like; non-ionic surfactants such as the propoxylated polyoxyethylene ethers, e.g., Poloxamer 231, Poloxamer 182, Poloxamer 184, and the like, the ethoxylated fatty acids, e.g., Tween 20, Myrj 45, and the like, the sorbitan derivatives, e.g., Tween 40, Tween 60, Tween 80, Span 60, and the like, the ethoxylated alcohols, e.g., polyoxyethylene (4) lauryl ether (Brij 30), polyoxyethylene (2) oleyl ether (Brij 93), and the like, lecithin and lecithin derivatives, and the like; the terpenes such as D-limonene, α-pinene, β-carene, α-terpineol, carvol, carvone, menthone, limonene oxide, α-pinene oxide, eucalyptus oil, and the like. Also suitable as skin penetration enhancers are organic acids and esters such as salicyclic acid, methyl salicylate, citric acid, succinic acid, and the like.

Embodiments of the invention may be practiced without the theoretical aspects presented. Moreover, the theoretical aspects are presented with the understanding that Applicant does not seek to be bound by the theory presented.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the embodiments described herein.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

### Example 1

### Acceleration of Wound Healing by Growth Hormone Releasing Hormone and its Agonists

### Materials and Methods

Peptides, Cell Culture, Western Blot and Histological Analysis.

hGHRH(1-29)NH₂ was obtained from Sigma. Agonist JI-38 was synthesized as reported (24, 26). The peptides were dissolved in phosphate buffered saline (PBS) at the indicated concentrations. Controls contained solvent alone. Fibroblasts were isolated using standard methods and maintained in Dulbecco's Modified Eagle Medium containing 10% FBS and antibiotics/antimycotics. For all experiments, primary fibroblasts of less than 10 passages old were used. For Western blot analysis, antibodies for αSMA, FAK and actin were obtained from Sigma while for SV1; the antibody 2317/5 was used (35). The same antibody was also used for the immunohistochemistry as previously described (36). Histology was performed on standard 5 μm sections of paraffin embedded material stained with hematoxylin/eosin. βGal-staining was performed in lung fibroblasts, isolated from transgenic mice bearing an αSMA-beta galactosidase reporter transgene. After treatment with the peptides for 24 hours, cells were washed (three times each for 5 min) with 0.1M PBS (pH 7.4), and fixed with 0.05% glutaraldehyde in PBS, for 10 min at room temperature. After three more washes, cells were stained with 1 mg/ml of 5-bromo-4-chloro-3indonyl β-D-galactopyranoside, 5 mM potassium ferrocyanide, 5 mM potassium ferricyanide and 2 mM MgCl₂) overnight at 37° C.

Cell Proliferation and Migration Assays.

The rate of cell proliferation was evaluated by the Trypan blue exclusion assay under inverted microscope. Cell migration was performed by the “scratch” assay as described, exposing cells to the peptides for 18 h (33). For the transwell-based migration assay, MEFs were seeded on 8 μm pore size transwells (Corning) and after 5 days exposure to the peptides at the indicated concentration, cell number was evaluated in the bottom compartment. All in vitro experiments were performed in triplicate and similar results were obtained.

Mice and Wound Healing Assay.

Wild-type mice of mixed C57BL6/FVB genetic background, originally obtained by Jackson Laboratories were maintained in our laboratory. Care of animals was in accord with University of Athens Institutional guidelines. The reporter αSMA-βGal mice have been described elsewhere (32). Wound healing assay in vivo was performed after administration of anesthesia (100 mg/kg ketamine, 10 mg/kg xylazine). Mice were shaved in the back, wiped with ethanol and wounds were cut using a biopsy punch (4 mm). The skin was grabbed at dorsal midline and placed over a cardboard backing on one side of the skin fold. The biopsy punch was pressed until both layers of the skin were punched and the cardboard was reached. Four wounds were performed per mouse, two on each side of the dorsal midline, at equal distance, so that the tension of the skin would be equal all over the mouse back and would not influence the contraction of the wounds. The experimental protocol was reviewed and approved by the University of Athens Animal Experimentation Committee. GHRH and JI-38 (100 nM) were applied twice daily in a volume of approximately 50 μl. At least 10 wounds for each of the control (vehicle, PBS), GHRH or JI-38 were observed. In order to avoid study variation due to the wound healing variability between different experimental animals, each mouse was its own control. For wound area measurements digital pictures of wounds were analyzed by the ImageJ (N1H) software. Wound size was monitored over time relative to a 4 mm diameter circular paper cutout placed next to the wound as previously described (38). For histology, 4-6 wounds per time point per group were analyzed. Similar results were obtained among different mice and histological sections for each group and representative are shown.

Van Gieson Elastic Stain for Collagen.

Tissue sections from mice wounds were de-paraffinized and rehydrated in distilled H₂O. These sections were then stained in orcein (Sigma) 1% w/v in 96% ethanol for 30 min at room temperature and washed in distilled H₂O. Then the sections were stained in Weigert's iron chloride solution (A: Ferric chloride 0.83% w/v, Ferrous sulphate 1.5% w/v, HCl 12N 0.67% v/v in distilled H₂O and B: Hematoxylin 1% w/v in absolute ethanol mixed in a proportion of 3A:1B) for 8 min at room temperature and washed in distilled H₂O. Finally sections were stained in picric-fuchsin solution (1.14% picric acid, 0.1% acid fuchsin in distilled H₂O) for 1 min at room temperature. Then slides were dehydrated and then mounted.

Statistical Analysis.

The data are presented as mean±SEM. Statistical analysis was performed by using 1-way ANOVA and Dunnett's test. The results were considered significant when p<0.05.

Growth Hormone-Releasing Hormone and its Agonistic Analogs:

These are shown in Table 1 below:

TABLE 1
Growth Hormone-Releasing Hormone GHRH(1-29)NH₂ and its agonistic analogs

| | Amino acid residue | | | | | | | | | | | | | | |
|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 |
| hGHRH | Tyr | Ala | Asp | Ala | Ile | Phe | Thr | Asn | Ser | Tyr | Arg | Lys | Val | Leu | Gly |
| (1-29)NH₂ H— | | | | | | | | | | | | | | | |
| | 1 | | | | | | | 8 | | | | 12 | | | 15 |
| hGH-RH | Tyr | | | | | | | Asn | | | | Lys | | | Gly |
| JI-34 | Dat | | | | | | | — | | | | Orn | | | Abu |
| JI-36 | Dat | | | | | | | Thr | | | | Orn | | | Abu |
| JI-38 | Dat | | | | | | | Gln | | | | Orn | | | Abu |

| | Amino acid residue | | | | | | | | | | | | | |
|---|---|---|---|---|---|---|---|---|---|---|---|---|---|---|
| | 16 | 17 | 18 | 19 | 20 | 21 | 22 | 23 | 24 | 25 | 26 | 27 | 28 | 29 |
| hGHRH | Gln | Leu | Ser | Ala | Arg | Lys | Leu | Leu | Gln | Asp | Ile | Met | Ser | Arg-NH₂ |
| (1-29)NH₂ H— | | | | | | | | | | | | | | |
| | | | | | | 21 | | | | | | 27 | 28 | 29 |
| hGH-RH | | | | | | Lys | | | | | | Met | Ser | Arg-NH₂ |
| JI-34 | | | | | | Orn | | | | | | Nle | Asp | Agm |
| JI-36 | | | | | | Orn | | | | | | Nle | Asp | Agm |
| JI-38 | | | | | | Orn | | | | | | Nle | Asp | Agm |

Non-coded amino acids are abbreviated as follows:
Dat: desaminotyrosine
Orn: ornithine
Abu: aminobutyric acid
Nle: norleucine
Agm: agmatine

### Results:

Mouse Embryonic Fibroblasts (MEFs) Express SV1.

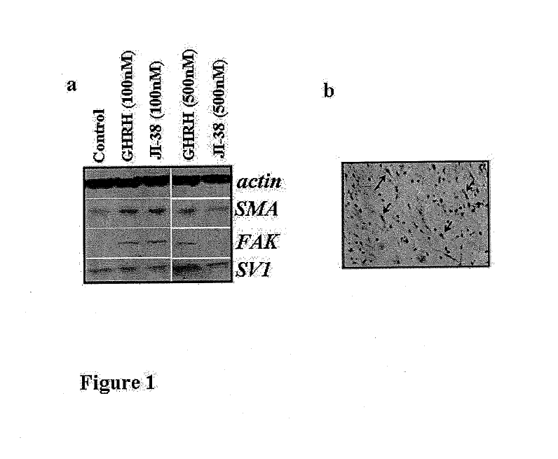

It was evaluated whether MEFs express the receptor for GHRH SV1. Primary MEFs were exposed to GHRH and agonistic GHRH analog JI-38 (24, 26) at 100 nM and 500 nM for 24 h and SV1 expression was then assessed by immunoblot analysis. As shown in FIG. 1A, MEFs expressed SV1 and the levels of SV1 expression were upregulated by GHRH at 500 nM. Immunohistochemical analysis of 4 mm-skin wounds, 5 days after skin wound incision revealed a mosaic pattern for anti-SV1 immunoreactivity confirming that at least a portion of fibroblasts within a dermal wound express SV1.

GHRH Induces the Expression of α-Smooth Muscle Actin (αSMA) in Fibroblasts.

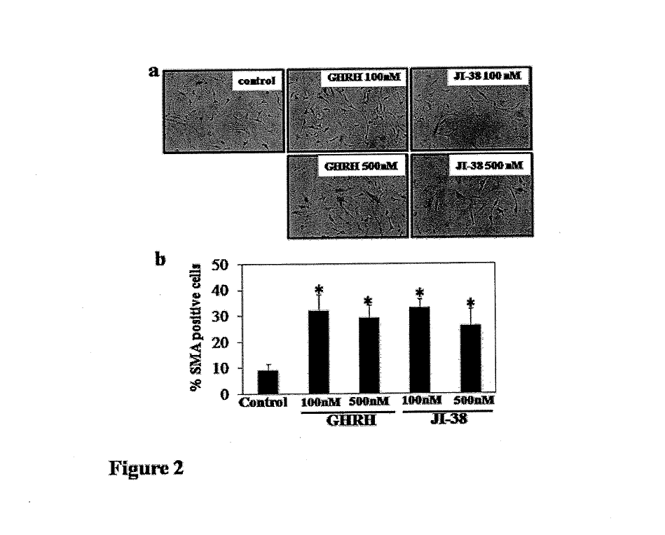

αSMA is an actin isoform that confers tensional and contractile activity (25,26). Typically SMA is selectively expressed in smooth muscle cells of adult animals, but is induced in activated fibroblasts (myofibroblasts) during wound healing, within the stroma of many tumors, and in general under conditions where there is extensive tissue reorganization and regeneration (27,28). Immunoblot analysis showed that GHRH and the agonist JI-38 stimulated SMA expression in MEFs (FIG. 1A). Of interest, this effect was significant at a concentration of 100 nM but not 500 nM of either peptide. A similar pattern of expression was found for focal adhesion kinase (FAK), an enzyme which plays a role in cell migration and invasion (29). The activation of αSMA in fibroblasts following GHRH treatment was confirmed in lung fibroblasts isolated from transgenic mice bearing a reporter β-galactosidase (βGal) transgene under the regulation of the αSMA promoter/enhancer (30). Cells from these mice display βGal positivity when αSMA expression is activated (30). As shown in FIG. 2, under normal culture conditions the fraction of βGal-positive cells is minimal, but following an exposure to GHRH or agonist 31-38, it is markedly increased about 3-fold compared to the untreated controls.

GHRH Promoted Cell Migration of MEFs In Vitro.

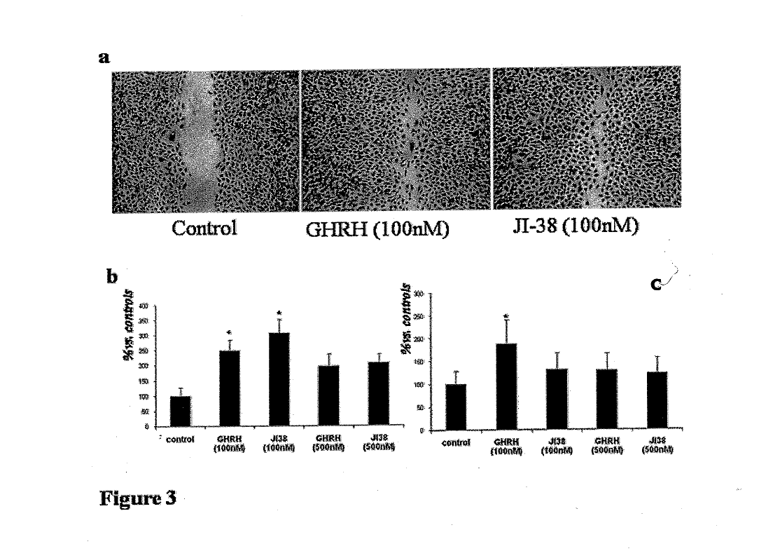

Subsequently MEFs were subjected to scratch wound migration assays in vitro, following exposure to GHRH and 31-38 at 100 nM. In a scratch assay (33), following exposure to GHRH and JI-38, migration of MEFs was considerably increased compared to the migration of control MEFs (FIG. 3A). Consistent with this finding, migration through 8 μm-pore transwells was significantly (p<0.05) increased in MEFs exposed to GHRH and 31-38 at 100 nM, by about 2.5- and 3 fold respectively, as compared to the controls (FIG. 3B). A considerable, notwithstanding not significant, stimulation of cell migration, by about 2-fold as compared to the controls, was also evidenced at 500 nM GHRH or 31-38. In order to rule out the possibility that the results of these two migration assays were simply due to the increased proliferation, MEFs were exposed to the peptide analogs and cell proliferation was evaluated. As shown in FIG. 3C, the proliferation of MEFs was stimulated following exposure to 100 nM GHRH while 500 nM GHRH and 100 nM and 500 nM JI-38 induced no considerable effect in the rate of cell proliferation.

GHRH and GHRH Agonist JI-38 Accelerated Wound Healing In Vivo.

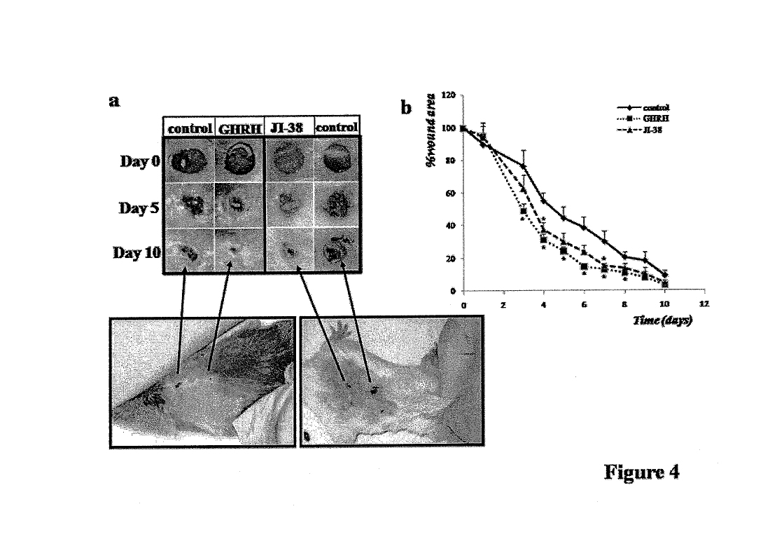

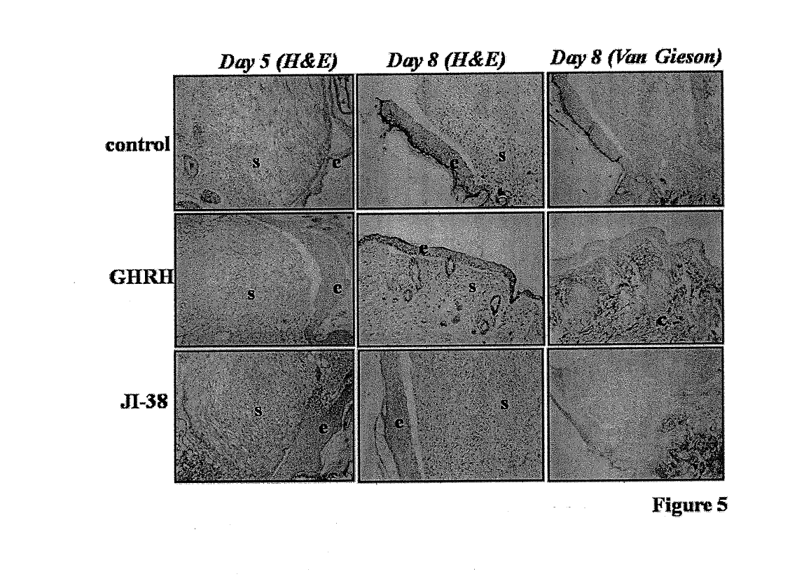

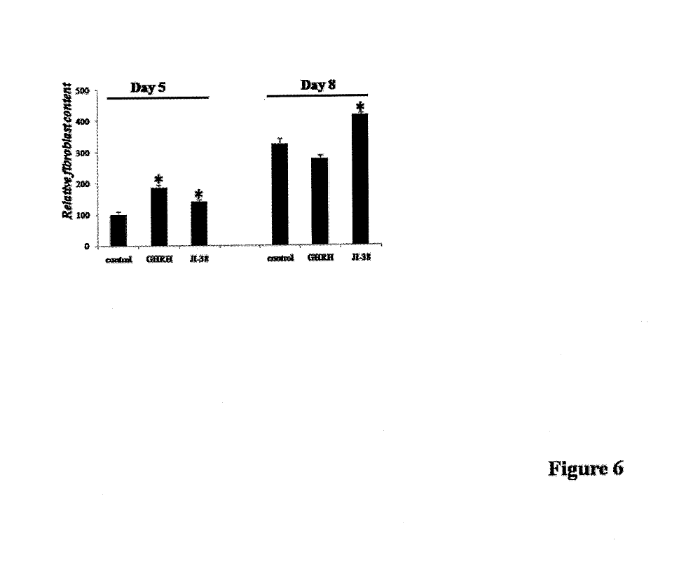

In order to evaluate the effect of GHRH on wound healing in vivo, wild type mice were subjected to 4 mm skin biopsies and the healing of the inflicted wounds in the presence or absence of GHRH and JI-38 at 100 nM was observed daily. In order to avoid variation in the wound healing process between different experimental animals, a control wound was always included in each mouse, thus permitting paired analysis. As shown in FIG. 4, a considerable acceleration of wound healing was observed in the presence of 100 nM GHRH or 31-38 as compared to the controls that were evidenced as soon as in day 3 and that resulted in nearly complete healing about 10 days post-incision (FIG. 4B). Histological analyses were performed on skin wounds at days 5 and 8 and showed increased content of fibroblasts, particularly in the wounds treated with GHRH, and to a lesser extent in the JI-38 treated wounds, as compared to the controls on day 5 (FIG. 5). Quantification of the fibroblast content in the wound area showed that fibroblast density was increased by about 80% and 40% in the GHRH and 31-38 treated wounds (p<0.05, FIG. 6), respectively. On day 8, reformation of the covering epithelium was almost complete following treatment with GHRH and skin resembled the normal, prior to the wound state. It was noted that both histologically and macroscopically GHRH was more potent than 31-38 at equimolar concentrations. At day 8, the epidermis had been regenerated in all 3 groups, but in GHRH-treated wounds the epidermis, as well as the dermis, were almost normal with a diffuse mild inflammatory infiltration. In JI-38 treated wounds the stroma was dense and rich in fibroblasts indicating a more advanced stage of healing as compared to the control (p<0.05). Van Gieson staining at day 8 (FIG. 5) indicated the nearly normally arranged collagen fibers in the lamina propria, of the GHRH treated samples. In the controls and JI-38 treated specimens, collagen fibers (red) could not be seen, implying delayed healing.

### Discussion

Wound healing is a complex process in which fibroblasts play an essential role. During healing, the orchestrated proliferation and migration of the resident dermal fibroblasts occurs and is followed by induction of expression of smooth muscle cell contractile proteins including αSMA, their modulation into myofibroblasts, and the re-organization of the extracellular matrix. This eventually results in skin tissue re-generation (29, 30, 34).

The principal action of the hypothalamic neuropeptide GHRH is considered to be the stimulation of synthesis and release of GH from the pituitary. However, there is increasing evidence implicating GHRH in a wide additional range of physiological and pathological processes including carcinogenesis, immune function, follicular maturation, Leydig cell differentiation, cardiac repair during myocardial infraction, and others (2, 9, 14-16). These findings, in combination with well-documented findings showing that there is extra-hypothalamic, local in situ production of GHRH by several peripheral tissues suggest a pleiotropic action for this peptide hormone with other targets remaining to be identified (2, 3). The present study provides fresh original evidence showing involvement of GHRH in dermal wound healing. Of particular significance, compelling evidence is provided herein showing that GHRH accelerated in vivo healing of skin wounds in mice. Moreover, results of these in vitro studies suggest that this may be due, at least in part, to augmentation of migration of wound-associated fibroblasts and activation of αSMA, which increases contractility of myofibroblasts, a process important in wound retraction and repair (30). Finally, evidence provided herein showing that fibroblasts express the GHRH receptor SV1 is significant because this identifies receptors that potentially mediate the direct effects of GHRH in this cell type.

Exposure of MEFs to GHRH or its agonistic analog JI-38 caused a stimulation in the expression of αSMA. This effect was greater at lower doses of GHRH and JI-38 (100 nM) and less pronounced at higher doses (500 nM). Since SV1 levels were similar or even stimulated by GHRH at 500 nM, it is not likely that the reduction in the potency of GHRH in stimulation of αSMA was due to the downregulation of SV1 receptors by the treatment. As evidenced by the βGal staining, the pattern of αSMA positivity was mosaic while the increase by the treatment was due to elevation of the fraction of the αSMA-positive cells, rather than the overall intracellular upregulation of αSMA in cultured cells. While this observation requires a validation by additional experiments, it is consistent with the previously reported transient stimulation of αSMA in experimental wounds (34).

By affecting proliferation (20), migration and αSMA expression, GHRH is implicated in various stages of the wound healing process. It is of interest that different doses of GHRH or agonist JI-38 did not elicit analogous effect in terms of the specific cellular response measured. The reasons for this are unclear, but may represent complex integration of multiple signaling pathways with differing sensitivities, differences in local concentrations of GHRH versus JI-38 secondary to differences in local solubility, systemic absorption, diffusion, binding properties, or tachyphylaxis as well as to differential receptor desensitization.

The fact that the agonist JI-38 was more potent than GHRH only at the transwell-based migration assay may be related to the fact that it was developed and characterized to be much more potent than GHRH, by using an in vivo growth hormone release assay after a subcutaneous administration (24,26). The high activity of JI-38 and related agonists was due to the resistance to degradation by subcutaneous peptidases (24, 26). The response to these GHRH agonists is based on the pituitary GHRH receptor (24, 26). It is therefore possible that receptors, such as SV1, that may mediate the effects of GHRH in the fibroblasts exhibit different sensitivity to the agonist than the pituitary type of GHRH receptor.

Collectively, our findings have identified a novel function for GHRH in the promotion of wound healing, wound and skin tissue repair, and wound and skin regeneration. Results evidence the possibility of therapeutic use of GHRH analogs for accelerating wound healing, tissue repair and tissue regeneration following traumatic injury, surgery, or disease.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such a feature may be combined with one or more other features of the other implementations as may be desired and advantageous for any given or particular application.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

### REFERENCES

1. Kovács M, Schally A V, Varga J L, Zarandi M. (2008) Endocrine and antineoplastic actions of growth hormone-releasing hormone antagonists. Curr Med Chem. 15:314-321.
2. Kiaris H, Schally A V, Kalofoutis A. (2005) Extrapituitary effects of the growth hormone-releasing hormone. Vitam Horm. 70:1-24
3. Kiaris H, Koutsilieris M, Kalofoutis A, Schally A V. (2003) Growth hormone-releasing hormone and extra-pituitary tumorigenesis: therapeutic and diagnostic applications of growth hormone-releasing hormone antagonists. Expert Opin Investig Drugs 12:1385-1394.
4. Schally A V, Varga J L, Engel J B. (2008) Antagonists of growth-hormone-releasing hormone: an emerging new therapy for cancer. Nat Clin Pract Endocrinol Metab. 4: 33-43.
5. Margioris N M, Brockmann G, Bohler J R H C L, Grino M, Vamvakopoulos N, Chrousos G P (1990) Expression and localization of growth hormone-releasing hormone messenger ribonucleic acid in rat placenta: in vitro secretion and regulation of its peptide product. Endocrinology 126: 151-158.
6. Berry S A, Pescovitz O H (1988) Identification of a rat GHRH-like substance and its mRNA in rat testis. Endocrinology 126: 661-663.
7. Moretti C, Fabbri A, Gnessi L, Bonifacio V, Bolotti M, Arizzi M, Nazzione Q, Spera G (1990) Immunohistochemical localization of growth hormone-releasing hormone in human gonads. J Endocrinol Invest 13: 301-305.
8. Stephanou A, Knight R A, Lightman S L (1991) Production of growth hormone-releasing hormone-like peptide and its mRNA by human lymphocytes. Neuroendocrinology 53: 628-633.
9. Ciampani T, Fabbri A, Isidori A, Dufau M L (1992) Growth hormone-releasing hormone is produced by rat Leydig cell in culture and acts as a positive regulator of Leydig cell function. Endocrinology 131: 2785-2792.
10. Bagnato A, Moretti C, Ohnishi J, Frajese G, Catt K J (1992) Expression of the growth hormone-releasing hormone and its peptide product in the rat ovary. Endocrinology 130: 1097-1102.
11. Bagnato A, Moretti C, Frajese G, Catt K J (1991) Gonadotropin-induced expression of receptors for growth hormone-releasing factor in cultured granulosa cells. Endocrinology 128: 2889-2894.
12. Moretti C, Bagnato A, Solan N, Frajese G, Catt K J (1990) Receptor-mediated actions of growth hormone releasing factor on granulosa cell differentiation. Endocrinology 127: 2117-2126.
13. Khorram O, Garthwaite M, Grosen E, Golos T (2001) Human uterine and ovarian expression of growth hormone-releasing hormone messenger RNA in benign and malignant gynaecological conditions. Fertility and Sterility 75: 174-179.
14. Khorram O, Garthwaite M, Golos T (2001) The influence of aging and sex hormones on expression of growth hormone-releasing hormone in the human immune system. J Clin Endocrinol Metab 86: 3157-3161.
15. Kanashiro-Takeuchi R M, Tziomalos K, Takeuchi L M, Treuer A V, Lamirault G, Dulce R, Hurtado M, Song Y, Block N L, Rick F, Klukovits A, Hu Q, Varga J L, Schally A V, Hare J M (2010) Cardioprotective effects of growth hormone-releasing hormone agonist after myocardial infarction. Proc Natl Acad Sci USA. 107:2604-2609.
16. Granata et al (2009) Growth hormone-releasing hormone promotes survival of cardiac myocytes in vitro and protects against ischaemia-reperfusion injury in rat heart. Cardiovasc Res 83:303-312.
17. Ludwig et al (2010) Agonist of growth hormone-releasing hormone as a potential effector for survival and proliferation of pancreatic islets. Proc Natl Acad Sci USA 107(28)12623-12628.
18. Rekasi Z, Czompoly T, Schally A V, Halmos G (2000). Isolation and sequencing of cDNAs for splice variants of growth hormone-releasing hormone receptors from human cancers. Proc Natl Acad Sci USA. 97:10561-10566.
19. Kiaris H, Schally A V, Busto R, Halmos G, Artavanis-Tsakonas S, Varga J L (2002) Expression of a splice variant of the receptor for growth hormone-releasing hormone (GHRH) in 3T3 fibroblasts activates cell proliferation responses to GHRH analogs. Proceedings of the National Academy of Sciences USA 99: 196-200.
20. Kiaris H, Chatzistamou I, Schally A V, Halmos G, Varga J L, Koutselini H, Kalofoutis A (2003) Ligand-dependent and -independent effects of GHRH receptor splice variant 1. Proceedings of the National Academy of Sciences USA 100: 9512-9517.
21. Kiaris H, Schally A V, Armatis P (2001) Direct action of growth hormone-releasing hormone agonist JI-38 on normal human fibroblasts: evidence from studies on cell proliferation and c-myc proto-oncogene expression. Regul Pept 96:119-24
22. Kiaris H and Schally A V (1999) Decrease in telomerase activity in U-87MG human glioblastomas after treatment with an antagonist of growth hormone-releasing hormone. Proceedings of the National Academy of Sciences USA 96:226-231.
23. Bellyei S, Schally A V, Zarandi M, Varga J L, Vidaurre I, Pozsgai E (2010). GHRH antagonists reduce the invasive and metastatic potential of human cancer cell lines in vitro. Cancer Lett 293: 31-40.
24. Schally A V and Comaru-Schally A M (1998) Growth Hormone Secretagogues In Clinical Practice, eds. Bercu B B, Walker R F (Dekker, New York), pp. 131-142.
25. McAnulty R J (2007) Fibroblasts and myofibroblasts: their source, function and role in disease. Int J Biochem Cell Biol. 39:666-671.
26. Izdebski J, Pinski J, Horvath J E, Halmos G, Groot K, Schally A V (1995) Synthesis and biological evaluation of superactive agonists of growth hormone-releasing hormone. Proc Natl Acad Sci USA 92:4872-4876.
27. B. Hinz, G. Celetta, J J Tomasek, G Gabbiani, C (2001). Chaponnier. Alpha-smooth muscle actin expression upregulates fibroblast contractile activity. Mol Biol Cell 12, 2730-2741.
28. Hinz B, and Gabbiani G (2003). Mechanisms of force generation and transmission by myofibroblasts. Curr. Opin. Biotechnol 14, 538-546.
29. A. Desmoulière, C. Guyot, G. Gabbiani (2004). The stroma reaction myofibroblast: a key player in the control of tumor cell behavior. Int J Dev Biol 48, 509-517.
30. Serini, G., and Gabbiani, G. (1999). Mechanisms of myofibroblast activity and phenotypic modulation. Exp Cell Res 250: 273-283
31. Hauck C R, Hsia D A, Schlaepfer D D (2002) The focal adhesion kinase—a regulator of cell migration and invasion. IUBMB Life. 53:115-119.
32. Tomasek J J, McRae J, Owens G K, Haaksma C J (2005) Regulation of alpha-smooth muscle actin expression in granulation tissue myofibroblasts is dependent on the intronic CArG element and the transforming growth factor-beta1 control element. Am J Pathol 166: 1343-1351.
33. Liang C C, Park A Y, Guan J L (2007) In vitro scratch assay: a convenient and inexpensive method for analysis of cell migration in vitro. Nat Protoc 2: 329-333.
34. Darby I, Skalli O, Gabbiani G (1990) Alpha-smooth muscle actin is transiently expressed by myofibroblasts during experimental wound healing. Lab Invest 63: 21-29.
35. Dorsett-Martin W. Rat models of skin wound healing: A review. (2004) Wound Rep Regen 12: 591-599.
36. Toller G L, Horvath J E, Schally A V, Halmos G, Varga J L, Groot K, Chism D, Zarandi M (2004). Development of a polyclonal antiserum for the detection of the isoforms of the receptors for human growth hormone-releasing hormone on tumors Proc Natl Acad Sci USA 101: 15160-15165.
37. Chatzistamou I, Volakaki A A, Schally A V, Kiaris H, Kittas C (2008) Expression of growth hormone-releasing hormone receptor splice variant 1 in primary human melanomas. Regul Pept 147: 33-36.
38. Gallucci R M, Simeonova P P, Matheson J M, Kommineni C, Guriel J L, Sugawara T, Luster M I (2000). Impaired cutaneous wound healing in interleukin-6-deficient and immunosuppressed mice. FASEB J. 14: 2525-2531.

1. A method of accelerating a healing process of cells, structures, and/or tissues in vivo comprising
exposing the cells, structures, and/or tissues to a therapeutically effective amount of growth hormone releasing hormone (GHRH) and/or an agonist of growth hormone releasing hormone (GHRH),
wherein the healing process is selected from the group consisting of repair, regeneration, and a combination, of the cells, structures, and/or tissues.

2. The method of claim 1, wherein the healing process is of a wound.

3. The method of claim 1, wherein the agonist of GHRH comprises a peptide set forth as SEQ ID NO: 1 and/or SEQ ID NO: 2.

4. The method of claim 3, wherein the agonist of GHRH comprises a peptide having the formula
Q¹-CO—R²—R³-Ala⁴-Ile⁵-Phe⁶-Thr⁷-R⁸-Ser⁹-Tyr¹⁰-Arg¹¹-R¹²—R¹³-Leu¹⁴-R¹⁵-Gln¹⁶-Leu¹⁷-Ser¹⁸-Ala¹⁹-Arg²⁰-R²¹—R²²—R²³-Gln²⁴-R²⁵-Ile²⁶-R²⁷—R²⁸—NH-Q²
wherein Q¹ is an omega or alpha-omega substituted alkyl having a structure 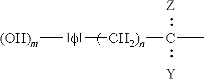
wherein IΦI is phenyl,
wherein Y is H, —NH₂, CH₃CONH— or CH₃NH—,
wherein Z is H or CH₃,
wherein m is 1 or 2 and n is 0, 1, or 2,
wherein R² is Ala, Abu or Aib,
wherein R³ is Asp or Glu,
wherein R⁸ is Asn, Ser, Gln or Thr,
wherein R¹² is Lys or Orn,
wherein R¹³ is Val or Ile,
wherein R¹⁵ is Ala, Gly or Abu,
wherein R²¹ is Lys or Orn,
wherein R²² is Leu, Ala or Abu,
wherein R²³ is Leu, Ala or Abu,
wherein R²⁵ is Asp or Glu,
wherein R²⁷ is Met, Nle, Ile, or Leu,
wherein R²⁸ is Asp, Asn or Ser,
wherein Q² is a lower omega-guanidino-alkyl group having a formula
(CH₂)ₚ—NH—C(NH₂)═NH, and
wherein p is 2-6;
or pharmaceutically acceptable salts thereof.

5. The method of claim 3, wherein the agonist of GHRH comprises a peptide having the formula
Q¹-CO-Ala²-Asp³-Ala⁴-Ile⁵-Phe⁶-Thr⁷-R⁸-Ser⁹-Tyr¹⁰-Arg¹¹-R¹²-Val¹³-Leu¹⁴-R¹⁵-Gln¹⁶-Leu¹⁷Ser¹⁸-Ala¹⁹-Arg²⁰-R²¹-Leu²²-Leu²³-Gln²⁴-Asp²⁵-Ile²⁶-R²⁷—R²⁸—NH-Q²
wherein Q¹-CO is Dat,
wherein R⁸ is Asn, Ser, Gln or Thr,
wherein R¹⁵ is Abu,
wherein at least one of R¹² and R²¹ is Orn,
wherein R²⁷ is Met or Nle,
wherein R²⁸ is Ser or Asp, and
wherein NH-Q² is Agm;
or pharmaceutically acceptable salts thereof.

6. The method of claim 3, wherein the agonist of GHRH is selected from the group consisting of JI-34 [Dat¹, Orn^{12,21}, Abu¹⁵, Nle²⁷, Asp²⁸, Agm²⁹]hGH-RH(1-29)NH₂, JI-36 [Dat¹, Thr⁸, Orn^{12,21}, Abu¹⁵, Nle²⁷, Asp²⁸, Agm²⁹]hGH-RH(1-29)NH₂, and JI-38 [Dat¹, Gln⁸, Orn^{12,21}, Abu¹⁵, Nle²⁷, Asp²⁸, Agm²⁹]hGH-RH(1-29)NH₂.

7. (canceled)

8. The method of claim 2, wherein the wound is selected from the group consisting of a surgical wound, an excisional wound, a deep wound, a skin graft, organ transplantation, tissue or organ damage, a soft tissue injury, a muscle tear, an eye tissue wound, a dental tissue wound, an oral cavity wound, a wound and/or ulcer of the gastro-intestinal mucosa, a diabetic ulcer, a dermal ulcer, a cubitus ulcer, an arterial ulcer, a venous stasis ulcer, a venous leg ulcer, a wound associated with ischemia and ischemic injury, a mechanical wound, an incision wound, a puncture wound, a tear wound, an abrasion wound, a projective wound, a burn, a thermal wound, a chemical wound, a radiation wound, a congestion-related wound, a traumatic wound, and a diabetes induced wound.

9. The method of claim 2, wherein the wound and/or the healing of the wound is effected or induced by a condition selected from the group consisting of uremia, malnutrition, vitamin deficiency, obesity, infection, immunosuppression, disease, a steroid, radiation therapy, an antineoplastic drug, an antimetabolite, a pharmaceutical, ischemia and ischemic injury.

10. (canceled)

11. The method of claim 1, comprising exposing the cells, structures, and/or tissues to an effective amount of tesamorelin.

12.-13. (canceled)

14. The method of claim 1, wherein the agonist of GHRH comprises a peptide set forth as SEQ ID NO: 1 and/or SEQ ID NO: 2.

15. The method of claim 1, wherein the agonist of GHRH is selected from the group consisting of JI-34 [Dat¹, Orn^{12,21}, Abu¹⁵, Nle²⁷, Asp²⁸, Agm²⁹]hGH-RH(1-29)NH₂, JI-36 [Dat¹, Thr⁸, Orn^{12,21}, Abu¹⁵, Nle²⁷, Asp²⁸, Agm²⁹]hGH-RH(1-29)NH₂, and JI-38 [Dat¹, Gln⁸, Orn^{12,21}, Abu¹⁵, Nle²⁷, Asp²⁸, Agm²⁹]hGH-RH(1-29)NH₂.

16. (canceled)

17. The method of claim 1, comprising administering a therapeutically effective amount of tesamorelin.

18. A method of inducing cell proliferation, cell migration, and/or smooth muscle actin-alpha (αSMA) expression in vitro and/or in vivo, comprising the step of exposing cells, structures, and/or tissues in vitro and/or in vivo to an effective amount of growth hormone releasing hormone (GHRH) and/or an agonist of growth hormone releasing hormone (GHRH).

19. The method of claim 18, wherein the agonist of GHRH comprises a peptide set forth as SEQ ID NO: 1 and/or SEQ ID NO: 2.

20. The method of claim 18, wherein the agonist of GHRH is selected from the group consisting of JI-34 [Dat¹, Orn^{12,21}, Abu¹⁵, Nle²⁷, Asp²⁸, Agm²⁹]hGH-RH(1-29)NH₂, JI-36 [Dat¹, Thr⁸, Orn^{12,21}, Abu¹⁵, Nle²⁷, Asp²⁸, Agm²⁹]hGH-RH(1-29)NH₂, and JI-38 [Dat¹, Gln⁸, Orn^{12,21}, Abu¹⁵, Nle²⁷, Asp²⁸, Agm²⁹]hGH-RH(1-29)NH₂.

21. (canceled)

22. The method of claim 18, comprising the step of exposing the cells, structures, and/or tissues to an effective amount of tesamorelin.

23.-27. (canceled)

28. A pharmaceutical composition comprising a therapeutically effective amount of growth hormone releasing hormone (GHRH) and/or an agonist of growth hormone releasing hormone (GHRH) for use in a method for accelerating a healing process of cells, structures, and/or tissues in vivo, wherein the healing process is selected from the group consisting of repair, regeneration, and a combination, of the cells, structures, and/or tissues.

29. The pharmaceutical composition of claim 28, wherein the healing process is of a wound.

30. (canceled)


SEQUENCE LISTING (9 sequences)

SEQ ID NO: 1 - protein, 27 aa, Artificial Sequence
  Description of Artificial Sequence Synthetic peptide
XXAIFTXSYRXXLXQLSARXXXQXIXX

SEQ ID NO: 2 - protein, 29 aa, Artificial Sequence
  Description of Artificial Sequence Synthetic peptide
YADAIFTXSYRXVLXQLSARXLLQDIXXR

SEQ ID NO: 3 - protein, 29 aa, Artificial Sequence
  Description of Artificial Sequence Synthetic peptide
YADAIFTNSYRXVLXQLSARXLLQDILDR

SEQ ID NO: 4 - protein, 29 aa, Artificial Sequence
  Description of Artificial Sequence Synthetic peptide
YADAIFTTSYRXVLXQLSARXLLQDILDR

SEQ ID NO: 5 - protein, 29 aa, Artificial Sequence
  Description of Artificial Sequence Synthetic peptide
YADAIFTQSYRXVLXQLSARXLLQDILDR

SEQ ID NO: 6 - protein, 44 aa, Artificial Sequence
  Description of Artificial Sequence Synthetic polypeptide
YADAIFTNSYRKVLGQLSARKLLQDIMSRQQGESNQERGARARL

SEQ ID NO: 7 - protein, 29 aa, Artificial Sequence
  Description of Artificial Sequence Synthetic peptide
YADAIFTNSYRXVLXQLSARXLLQDILSR

SEQ ID NO: 8 - protein, 29 aa, Artificial Sequence
  Description of Artificial Sequence Synthetic peptide
YADAIFTNSYRKVLGQLSARKLLQDIMSR

SEQ ID NO: 9 - protein, 29 aa, Artificial Sequence
  Description of Artificial Sequence Synthetic peptide
YADAIFTNSYRKVLGQLSARKLLQDIMSR